(19) United States
(12) Patent Application Publication
(10) Pub. No.: US 2025/0235576 A1
(43) Pub. Date: Jul. 24, 2025

(54) FIXTURE FOR ONE OR MORE ION GENERATOR DEVICES

(71) Applicant: Air+, Inc., Rye, NY (US)

(72) Inventor: Lawrence T. Sunshine, Rye Brook, NY (US)

(21) Appl. No.: 19/029,349

(22) Filed: Jan. 17, 2025

Related U.S. Application Data
(60) Provisional application No. 63/622,932, filed on Jan. 19, 2024.

(51) Int. Cl. A61L 9/22

(52) U.S. Cl. CPC A61L 9/22; A61L 2209/11; A61L 2209/12; A61L 2209/15; A61L 2209/16

(57) ABSTRACT

A fixture configured to retain one or more ion generator devices is provided. The fixture comprises a first wall, a second wall opposing the first wall and a connecting wall connecting the first wall and the second wall. The first wall, the second wall or the connecting wall has one or more openings. When one or more ion generator devices is retained in the fixture, the first wall and the second wall sandwich the one or more ion generator devices and light emitted or not emitted from a status indicator of a respective ion generator device is viewable from outside the fixture via a corresponding opening.

### CROSS REFERENCE TO RELATED APPLICATIONS

This application claims the benefit of U.S. Provisional Application No. 63/622,932 filed on Jan. 19, 2024, which is incorporated by reference herein in its entirety.

### FIELD OF THE DISCLOSURE

The present disclosure relates to ion generator devices and more particularly to a fixture which may hold one or more ion generator devices. The fixture may be mounted to or in an air conduit within an air flow system such as a heating, ventilating, and air-conditioning system.

### BACKGROUND

Air flow systems typically comprise one or more elements which move and/or treat air. An air flow system may treat air by heating or cooling the air. The air circulating within an air flow system and the space served by the air flow system may be contaminated with foreign contaminants such as particulates, microorganisms and volatile organic compounds. An air flow system may also treat the air by neutralizing the contaminants (e.g., decontaminate).

Ionizers, also known as ion generator devices, may be used to decontaminate air circulating within an air flow system and the space served by the air flow system. Certain ionizers may include high voltage electrodes. In order to decontaminate the air circulating within the air flow system and the space served by the air flow system, one or more ion generator devices may need to be mounted within the air flow system such that the produced ions may flow into the air. For example, the ion generator devices may be mounted within or to an air conduit of the air flow system.

However, depending on the size of the air conduit, mounting one or more ion generator devices may be difficult. Additionally, once the ion generator devices are mounted within or to the air conduit, it may be difficult to determine whether one of the ion generator devices has failed and needed to be replaced. It may also be difficult to nearly impossible to replace a single ion generator device.

### SUMMARY

Accordingly, disclosed is a fixture configured to retain one or more ion generator devices. The fixture comprises a first wall, a second wall opposing the first wall and a connecting wall connecting the first wall and the second wall. The first wall, the second wall or the connecting wall has one or more openings. When one or more ion generator devices is retained in the fixture, the first wall and the second wall sandwich the one or more ion generator devices and light emitted or not emitted from a status indicator of a respective ion generator device is viewable from outside the fixture via a corresponding opening.

In an aspect of the disclosure, the connecting wall may have the openings.

In an aspect of the disclosure, the connecting wall may be inclined with respect to the second wall, such as being inclined by about 45°.

In an aspect of the disclosure, the fixture may further comprise a protective film or lens inserted into each opening.

In an aspect of the disclosure, the first wall, the second wall and the connecting wall may provide a channel configured for electrical wiring to be routed from an external control panel to each of the one or more ion generator devices.

In an aspect of the disclosure, the fixture is configured to be mounted in a first orientation or a second orientation, where the first orientation is different from the second orientation. To facilitate mounting, in an aspect of the disclosure, the fixture comprises a plurality of mounting brackets. When the fixture is mounted in the first orientation, the plurality of mounting brackets is in a first position and when the fixture is mounted in the second orientation, the plurality of mounting brackets is moved to a second position. In an aspect of the disclosure, the fixture is configured to be mounted in one of the first orientation or the second orientation downstream of a heating or cooling coil of a heating, ventilating, and air-conditioning system (HVAC). In an aspect of the disclosure, the fixture may be mounted in an air handling unit (AHU), an air duct, a rooftop unit (RTU), a dedicated outdoor air system (DOAS), packaged Terminal Air Conditioners (PTAC) or a makeup air unit (MAU).

In an aspect of the disclosure, the fixture may retain a plurality of ion generator device where they are aligned. In an aspect of the disclosure, an ion generator device may generate positive ions, negative ions or both positive and negative ions. For example, each ion generator device may comprise two electrodes: a first electrode for the positive ions and a second electrode for the negative ions. In an aspect of the disclosure, when mounted in the fixture, adjacent ion generator devices may be in different orientations such that a first electrode is adjacent to another first electrode or a second electrode is adjacent to another second electrode.

In an aspect of the disclosure, the fixture may further comprise a first endcap and a second endcap positioned at respective ends in a longitudinal direction. The first endcap may have an opening configured to receive electrical wiring to be routed to each of the one or more ion generator devices. The electrical wiring is connected to an external control panel. The external control panel may comprise a power source. In an aspect of the disclosure, a strain relief fitting may be inserted into the opening of the first endcap. The strain relief fitting may surround the electrical wiring.

In an aspect of the disclosure, the fixture may further comprise a sectional fourth wall extending between the first wall and the second wall and between the one or more ion generator devices. The sectional fourth wall may extend from the first wall to the second wall.

In an aspect of the disclosure, the first wall, the second wall and the connecting wall may be formed from a single sheet of material.

In an aspect of the disclosure, a length of the fixture in a longitudinal direction may be between about 1 foot to about 12 feet.

Also disclosed is a fixture comprising a first wall, a second wall opposing the first wall and a connecting wall connecting the first wall and the second wall. The fixture is configured to retain one or more ion generator devices. The first wall, the second wall or the connecting wall have one or more openings and one or more status indicators. When one or more ion generator devices is retained in the fixture, the first wall and the second wall sandwich the one or more ion generator devices, and each of the one or more ion generator devices is connected to a corresponding one of the status indicators by a respective connection cable routed through an opening. All connection cables may be routed through a single opening or each connection cable may be routed through a different opening.

In other aspects of the disclosure, the one or more status indicators may be mounted into one or more openings in a wall of the fixture.

### DESCRIPTION

### Brief Description of Drawings

FIG. 1 illustrates a rear perspective view of a fixture for one or more ion generator devices in accordance with aspects of the disclosure;

FIG. 2 illustrates a front perspective view of the fixture in accordance with aspects of the disclosure;

FIG. 3 illustrates an end view of the fixture in accordance with aspects of the disclosure;

FIG. 4B illustrates a cross-sectional view of the fixture in accordance with aspects of the disclosure, taken along line A-A in FIG. 4A, where the cross-sectional view is taken through one of the openings;

FIG. 5 illustrates a top view of the fixture in accordance with aspects of the disclosure;

FIG. 6A illustrates the fixture mounted to an air conduit upstream of the heating or cooling coil in a first orientation in accordance with aspects of the disclosure;

FIG. 6B illustrates the fixture mounted to an air conduit upstream of the heating or cooling coil in a second orientation in accordance with aspects of the disclosure;

FIG. 7A illustrates the fixture mounted to an air conduit downstream of the heating or cooling coil in a first orientation in accordance with aspects of the disclosure;

FIG. 7B illustrates the fixture mounted to an air conduit downstream of the heating or cooling coil in a second orientation in accordance with aspects of the disclosure;

FIG. 8A illustrates a plurality of fixtures mounted to an air conduit upstream of the heating or cooling coil in a first orientation in accordance with aspects of the disclosure;

FIG. 8B illustrates a plurality of fixtures mounted to an air conduit downstream of the heating or cooling coil in a first orientation in accordance with aspects of the disclosure;

FIG. 9 illustrates an orientation of the adjustable mount for a second orientation mount; and

FIG. 10 illustrates a front perspective view of another example of a fixture with a sectional fourth wall in accordance with aspects of the disclosure,

### DETAILED DESCRIPTION

Aspects of the disclosure provide a fixture 1 for holding one or more ion generator devices 20 which enables visual inspection of a status indicator 100 of each ion generator device 20. This visual inspection, from external to the fixture 1, allows a determination of whether an ion generator device 20 has failed and needs to be replaced. In response to a detection of a faulty ion generator device, the faulty ion generator device may be removed from the fixture 1 and replaced with a new ion generator device without a need to dismantle the fixture or remove all of the ion generator devices 20 for testing to determine which ion generator device failed.

Aspects of the disclosure also provide a fixture 1 which may be mounted to an air conduit 605 of an air flow system in multiple orientations. The different mounting orientations provide a fixture 1 with versatility such that even where the space available for mounting may be narrow, the fixture 1 may be positioned within or on the air conduit 605 to enable the ions to flow within the air flow system and the spaced served by the air flow system.

The air flow system may be a heating, ventilating, and air-conditioning system (HVAC). The air conduit 605 may be any structure forming an enclosed air channel for air to flow within the air flow system. For example, the air conduit 605 may be a structure such as, but not limited to an air handling unit (AHU), an air duct, a rooftop unit (RTU), a dedicated outdoor air system (DOAS), packaged Terminal Air Conditioners (PTAC) or a makeup air unit (MAU). The air conduit 605 may also be a fan coil unit (FCU). The air conduit 605 may also be a wall mount unit or a ceiling cassette.

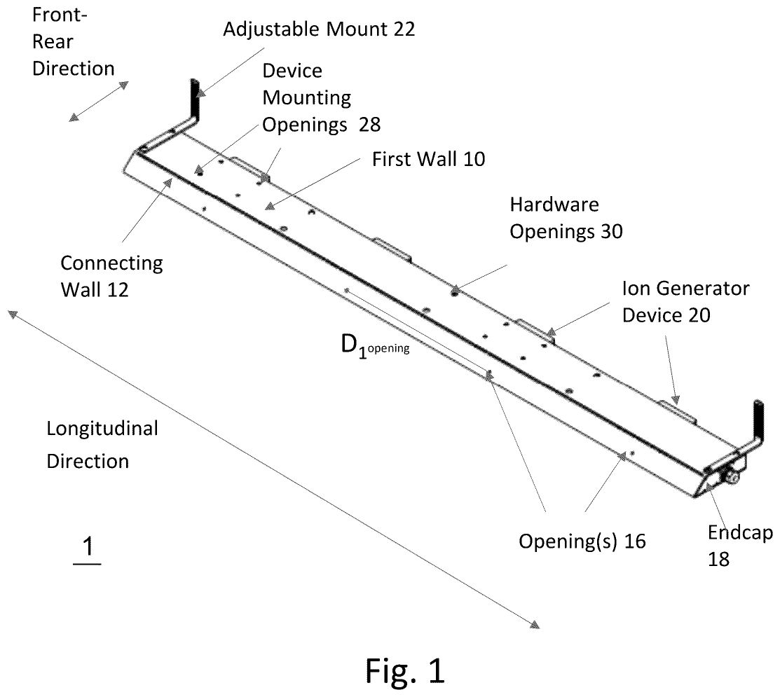

FIG. 1 illustrates a rear perspective view of a fixture 1 configured to hold one or more ion generator devices 20 in accordance with aspects of the disclosure. The fixture 1 may have three sides (walls) (not including the endcaps 18) (all of the sides are not shown in FIG. 1). The number of walls described herein refers to walls extending in a longitudinal direction. However, the fixture 1 is not limited to being only three walls (not including the endcaps 18). The fixture 1 may have two walls, four walls, or more.

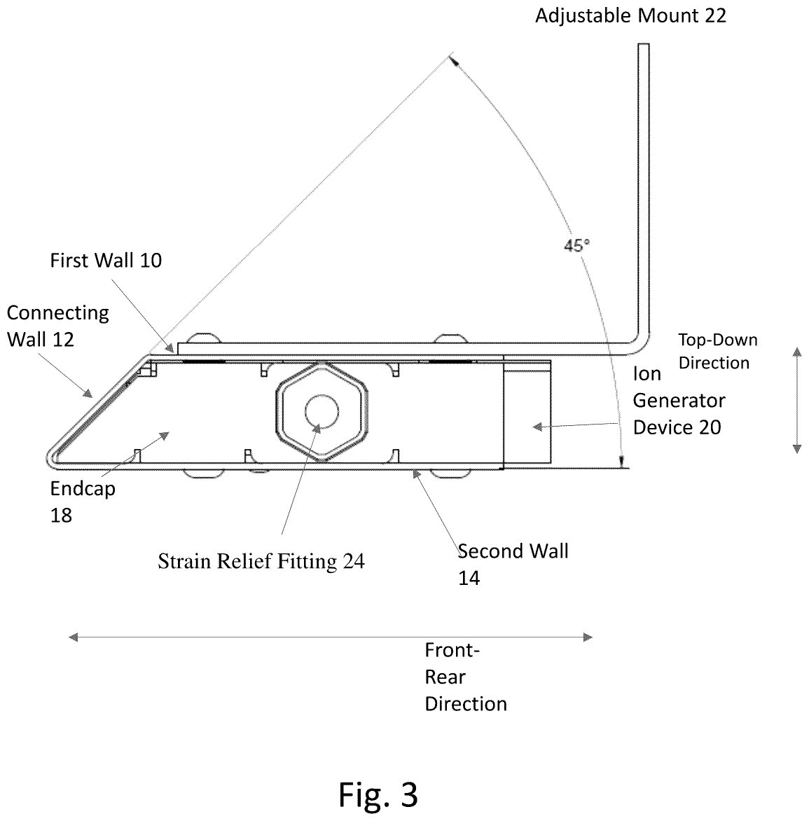

The fixture 1 has a first wall 10, a second wall 14 and a connecting wall 12. The second wall 14 is spaced apart from the first wall 10 in a top-down direction (such as shown in FIG. 3). The top-down direction is an example of a first direction. The second wall 14 opposes the first wall 10. The length of the space between the first wall 10 and the second wall 14 in the top-down direction may be based on the type of the ion generator device 20 to be held by the fixture 1. The first wall 10 and the second wall 14 sandwich the ion generator device 20.

The connecting wall 12 connects the first wall 10 and the second wall 14. As depicted in the figures, the connecting wall 12 is inclined. In some aspects, the connecting wall 12 may be linear, e.g., linearly inclined. The inclination provides a smooth transition for the air to flow around the fixture 1. This reduces any mechanical turbulence in the flow path of the air flow causes by the fixture 1. The reduction in the turbulence enables the air flow to maintain a substantially laminar air flow within the air conduit 605, when the fixture 1 is mounted in a horizontal orientation.

Additionally, when the fixture 1 is mounted in the air conduit 605 in a horizontal orientation, the lengths of the paths the air flows around the fixture 1 are different between the top (e.g., first wall side) and the bottom (e.g., second wall side) due to the inclination. For example, a path formed by the inclined wall (connecting wall 12) and the first wall 10 is longer than a path formed by the second wall 14. The difference may lead to a pressure difference and different air flow speeds depending on the relative position with respect to the fixture 1. This difference may help distribute the ions which are generated by the ion generator devices 20 within the air conduit 605. For example, in a case where the fixture 1 is mounted at the top of the air conduit 605 in a horizontal orientation with the first wall 10 facing upward, the difference in the flow may push the generated ions downward in the air conduit 605 such that the ions are spread lower within the air channel. Additionally, in case where the fixture 1 is mounted at the bottom of the air conduit 605 in a horizontal orientation with the first wall 10 facing downward, the difference in the flow may push the generated ions upward in the air conduit 605 such that the ions are spread higher within the air channel.

In some aspects, the angle of inclination of the connecting wall 12 may be about 45° (e.g., angle between the second wall 14 and the connecting wall 12) such as shown in FIG. 3. In some aspects, the angle may be based on the thickness of the ion generator devices 20 (in the top-down direction). For example, when the ion generator devices 20 are thicker, the angle may be larger and when the ion generator devices 20 are thinner, the angle may be smaller.

In other aspects of the disclosure, instead of the connecting wall 12 being substantially linear, the connecting wall 12 may be non-linear such as having a convex shape (curved), which also creates a smooth path for the air to flow.

In other aspects, the connecting wall 12 may be perpendicular to both the first wall 10 and the second wall 14.

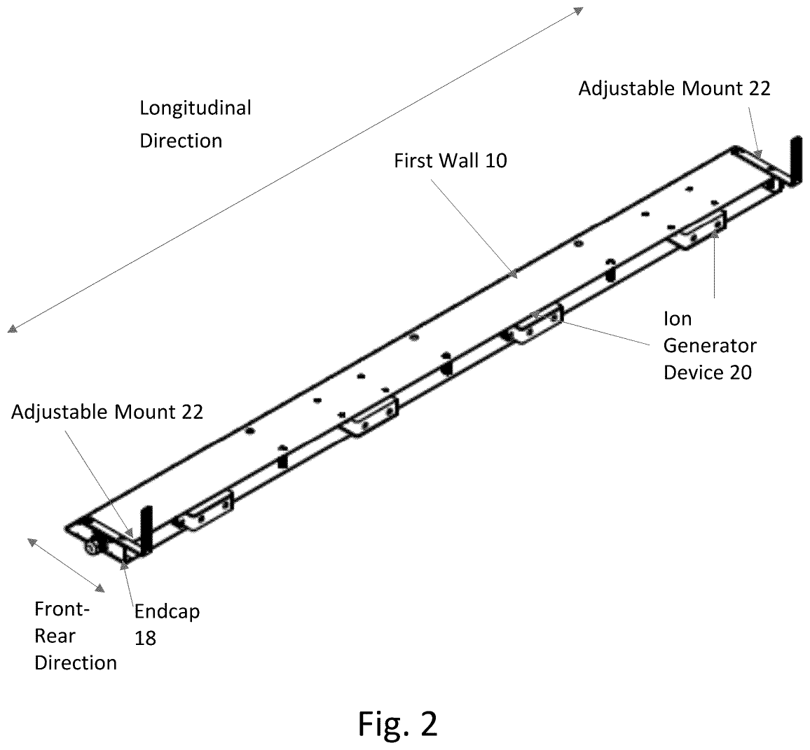

The first wall 10, the connecting wall 12 and the second wall 14 provide a compartment for one or more ion generator devices 20. In some aspects, the ion generating element(s) of the ion generator device 20 face the open end of the compartment. In other words, the ion generating element(s) is exposed as shown in FIG. 2.

Each ion generator device 20 may have ion generating element(s) configured to produce positive ions or positive ions. In some aspects, each ion generator device 20 can produce both positive and negative ions. The ion generator device 20 may produce both positive or negative ions at the same time or different times. The ion generating elements(s) may be any element capable of produces ions such as, but not limited to, ionizing needles, tubes, brushes, etc., Each needle may have a rod shape and the distal end may be exposed from the compartment. When an ion generator device 20 has two electrodes, the electrodes may alternate in time producing positive ions and negative ions. In other aspects, a single electrode may be controlled to produce positive ions and negative ions at different times, e.g., alternate.

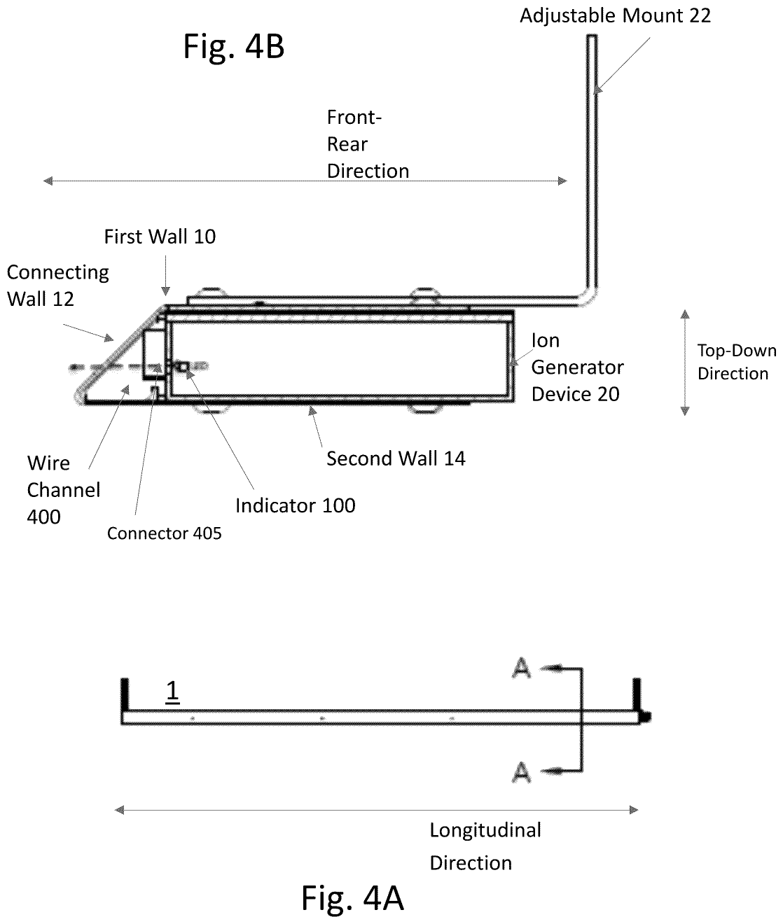

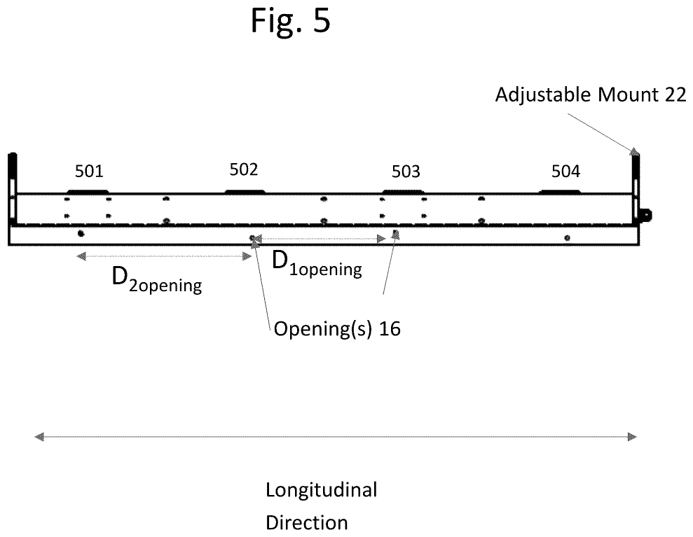

In some aspects of the disclosure, each ion generator device 20 may have a portion extending beyond the front edge of the first wall 10 and the second wall 14 in a front-rear direction as shown in FIG. 4B (see also FIG. 5). This portion contains the ion generating elements. Since the portion extends beyond the front edge of the first wall 10 and the second wall 14 in the front-rear direction, it reduces a likelihood that the generated ions come in contact with the walls 10, 14 prior to interacting with a contaminant. The front-rear direction is an example of a second direction.

Each ion generator device 20 may have a casing. The casing may have mounting tabs for mounting the ion generator device 20 to the fixture 1. The first wall 10 and/or the second wall 14 may have device mounting openings 28 corresponding to an area where the mounting tabs are in the casing (when the ion generator device 20 is disposed in the fixture 1) for mounting.

In some aspects, the casing of each ion generator device 20 may have a status indicator 100 which indicates an operation status of the ion generating element(s) (and/or the ion generator device 20). In some aspects, the status indicator 100 may be a light emitting diode (LED). The LED may emit a light when the ion generating elements are malfunctioning. In other aspects, the LED may emit a light when the ion generating elements are functioning. In yet other aspects, the LED may emit or not emit light when any element of the ion generator device 20 is operational or malfunctioning. The LED indicates (either via light or the absence of light) whether the ion generator device 20 needs to be replaced.

Since, the fixture 1 is mountable to an air conduit 605 and the fixture 1 may have multiple ion generator devices 20, the fixture 1 may have one or more openings 16 corresponding each ion generator device 20. The openings 16 may be positioned in proximity to the status indicator 100 to enable the light emitted from the LED or absence of light emitted from the LED to be viewable from outside of the fixture 1. In some aspects, when the ion generator devices 20 are mounted in the fixture 1, the openings 16 are aligned with the status indicator 100 for the ion generator device 20. In other aspects, the openings may be offset, however, the light or absence of light may be still viewable from outside.

In some aspects, the openings 16 may also enable cooling of each ion generator device 20, respectively.

In other aspects, each opening 16 may have a transparent film or lens covering the opening. The transparent film or lens transmits wavelengths of light overlapping the wavelength of the LED. The transparent film or lens may be used to prevent debris (including moisture) from entering the fixture 1 via the opening 16. Further, the transparent film or lens may have an etched, fronted or rough outer surface to diffuse the existing light emitted from the LED. This diffusion or light scattering makes the light visible at a greater angle from the outside of the fixture 1. Advantageously, the lens may be used where the status indicator 100 is not aligned with the opening, which provides a greater flexibility in the offset between a status indicator 100 and the opening 16 and allows the light or absence of light to be more easily viewed from outside the fixture.

In some aspects, as shown in FIGS. 1 and 5, the openings 16 may be in the connecting wall 12. However, the openings 16 may be in the first wall 10 or the second wall 14. For example, the location of the openings may depend on the location of the status indicator 100 on the casing of the ion generator device 20.

In some aspects of the disclosure, the distance between openings 16 in the longitudinal direction may be different such as shown in FIG. 5 (D_{1opening} V. D_{2opening}). The longitudinal direction is an example of a third direction. In some aspects, the status indicator 100, in each ion generator device 20, may not be symmetric on the casing of the ion generator device 20 in the longitudinal direction when the ion generator device is installed into the fixture 1. Therefore, the disposition of the openings 16 may be customized for the position of the status indicator 100 on the casings (in the longitudinal direction when the ion generator device is installed into the fixture 1). For example, D_{2opening} (distance between a first set of adjacent openings) may be larger than the distance between a second set of adjacent openings (D_{1opening}).

Additionally, in some aspects of the disclosure, the openings 16 may not be vertically aligned in the top-down direction. As will be described later, the orientation of adjacent ion generator devices 20 may be reversed. The status indicator 100 for each ion generator device may also not be vertically symmetric (in the top-down direction when installed in the fixture 1) and therefore, the alternate orientation of adjacent ion generator devices 20 may change the vertical position of the status indicator 100 in the top-down direction. In this aspect of the disclosure, the position of the opening may change vertically to maintain proximity to the status indicator 100 as shown in FIG. 5.

For example, FIG. 4B shows a cross-section of the fixture 1 through A-A (in FIG. 4A). The dash line in FIG. 4B shows the direct path from the status indicator 100 to the opening 16.

In some aspects of the disclosure, the orientation of adjacent ion generator devices 20 may be reversed to reduce the re-combination of positive ions generated by a first ion generator device and negative ions generated by a second ion generator device when the ion generator devices 20 are configured to generated both positive ions and negative ions. Since an ion has a charge (either positive or negative), there is a chance that the ion may recombine with the opposite charged ion prior to combining with a contaminant. Thus, adjacent ion generator devices 20 may be positioned within the fixture 1 such that the ion generating elements which generate the positive ions are adjacent to each other and the ion generating elements which generate the negative ions are adjacent to each other. This alternate orientation is shown in FIGS. 1, 2, and 5. This may result in the device mounting openings 28 alternating, e.g., in either the first wall 10 (such as shown in FIGS. 1, 2, and 5) or the second wall 14. For example, as shown in FIG. 5, the device mounting openings 28 for ion generator device 501, 503 are in the first wall 10, wherein the device mounting openings 28 for ion generator device 502, 504 are in the second wall 14.

In some aspects of the disclosure, where the ion generator devices 20 have a single electrode to produce both the positive and the negative ions (at different timing). The timing that the ion generator devices 20 produce the positive and negative ions may be controlled to reduce the re-combination of the positive ions with the negative ions. For example, each ion generator device may be controlled to produce positive ions in synchronization and produce negative ions in synchronization. Similarly, in a case where the ion generator devices 20 have multiple electrodes, where the same electrode can produce positive or negative ions or different electrodes can produce positive ions or negative ions (at certain times), the timing may be controlled to reduce the re-combination of the positive ions with the negative ions.

In an aspect of the disclosure, instead of the status indicator 100 being integral with the ion generator device 20, such as on the casing, each ion generator device 20 may have a remote status indicator. For example, a status indicator corresponding to each of the ion generator device(s) 20 may be mounted on a surface of one of the walls 10, 12, or 14. In this aspect of the disclosure, a cable connecting a respective ion generator device 20 and its corresponding status indicator may extend from the respective ion generator device 20 through a respective opening 16 in a wall of the fixture to the corresponding status indicator. A surface mounted status indicator may be easily viewed from outside the fixture 1 to indicate which ion generator device(s) has malfunctioned and/or needs maintenance. In some aspects of the disclosure, the surface mounted status indicator may be adjacent to the corresponding ion generator device 20. In other aspects of the disclosure, one of the walls 10, 12, or 14, may have a single opening 16. In this aspect, cables connected to a plurality of ion generator devices 20, respectively, may be routed from the plurality of ion generator devices 20 to a plurality of surface mounted status indicators, respectively, via a single opening 16. The cables may be routed using the wire channel 400.

In some aspects of the disclosure, each status indicator may be identified with a unique identifier to distinguish between the different ion generator device(s) 20. For example, each status indicator may be labeled with a number or letter identifying a specific ion generator device(s) 20. In other aspects, each ion generator device 20 may be identified by a unique light color (such as a unique LED color).

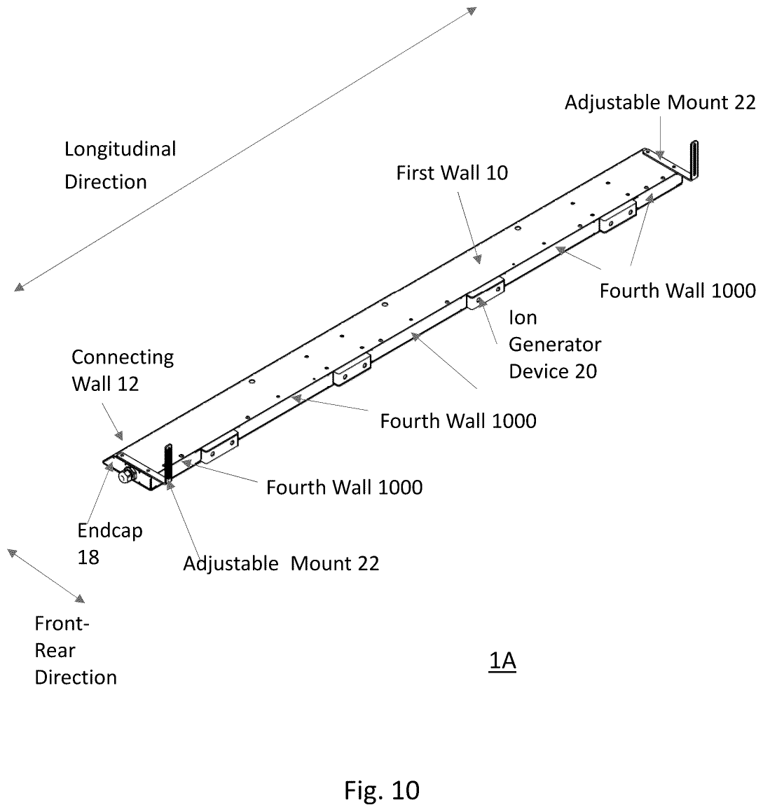

In other aspects of the disclosure, in a case where the fixture has a sectional fourth wall 1000 (as shown in FIG. 10), the remote status indicator may be mounted on the surface of the sectional fourth wall 1000. For example, each remote status indicator may be mounted in the section adjacent to a corresponding ion generator device 20.

In other aspects of the disclosure, the remote status indicator may be mounted within the opening(s) 16 of one of the walls 10, 12 or 14 (such as a panel mount indicator). In other aspects, in a case where the fixture has a sectional fourth wall 1000, the sectional fourth wall 1000 may comprise openings 16 and the remote status indicator may be mounted within the openings 16 of the section fourth wall 1000.

In some aspects of the disclosure, the first wall 10, the connecting wall 12 and the second wall 14 of the fixture 1 may be made of a single sheet of material stamped into a target compartment shape (other than the endcap(s)). The material may be a flexible material such that it is bendable to form the shape of the compartment yet also once formed, may be able to maintain the target compartment shape. The material may have such resiliency to return to the target compartment shape even after the walls 10, 14 may be intentionally moved such as for installation of the ion generator devices 20 or removal and replacement. In some aspects, the first wall 10, the connecting wall 12 and the second wall 14 may be made of a metallic material such as, but not limited to, aluminum and steel (stainless, galvanized). In other aspects, the walls 10, 12, 14 may be made of a polymer.

In an aspect of the disclosure, the first wall 10, the connecting wall 12 and the second wall 14 are dimensioned such that when each ion generator device 20 is mounted in the fixture 1, there is a gap or wire channel 400 (wire channel) between the connecting wall 12 and each ion generator device 20. The wire channel 400 extends in the longitudinal direction. For example, when the ion generator devices 20 are mounted, the rear end of the casing of each ion generator device 20 may be substantially aligned with the rear edge of the first wall 10 (as shown in FIG. 4B) in the front-rear direction. When the connecting wall 12 is inclined, the length of the first wall 10 is shorter than the length of the second wall 14 in the front-rear direction. The wire channel 400 is formed from a portion of the second wall and the connecting wall 12.

The wire channel 400 provides a space for electrical wires (wiring) to be connected from outside the fixture to one or more connectors 405 of the ion generator device. Each ion generator device 20 may have a power connector and a data connector (collectively connectors 405). The power connector is for receiving power from an external power source. The external source may provide a fixed current or fixed voltage as the source. In some aspects, the voltage may be a high voltage (current) to generate the ions. In other aspects, the voltage may be a low voltage (current) to power internal circuitry within the ion generator device 20.

The fixture 1 may be connected to a control panel (not shown). The control panel may include the external source. The control panel may include an AC or DC supply. The specific voltage and/or current supply (including either AC or DC) may be based on the specific ion generator devices 20 used in the fixture 1. One end of the wire(s) may be connected with the control panel and the other end may be connected to the connectors 405 of each ion generator device 20.

One or both ends (in the longitudinal direction) of the fixture 1 may have endcaps 18. The endcaps 18 prevent debris and moisture from entering the fixture 1. The endcaps 18 also provide further structural stability for the ends (in the longitudinal direction) of the fixture 1. This may be particularly useful where the longitudinal length is greater than a certain length.

In an aspect of the disclosure, the endcaps 18 may be the same material as the walls 10, 12, 14 such as a metallic material including, but not limited to, aluminum and steel (stainless, galvanized). In other aspects of the disclosure, the endcaps 18 may be made of a different material. In some aspects, the endcaps 18 may be removably attached to the walls 10, 12, 14 such as via a friction fit to facilitate maintenance such as replacement of one or more of the ion generators devices 20. In other aspects, similar to the walls, 10, 12, 14, the endcaps may be fabricated via stamping.

In an aspect of the disclosure, one of the endcaps 18 has an opening for the wiring. A strain relief fitting 24 may be inserted into the opening. The strain relief fitting 24 provides protection for the wires from it sheathing from being cut and protects the wires from stress and damage. The strain relief fitting 24 also prevents the wires from movement by securing the same to the fixture 1. In some aspects, the strain relief fitting 24 may be made of an elastomer material.

In an aspect of the disclosure, the ion generator devices 20 may be installed into the fixture 1 by rotating the one or both the first wall 10 and second wall 14 to provide space to insert the ion generator devices 20 in the open end. The ion generator devices 20 may then be inserted into the fixture 1, whereby the mounting tabs of each ion generator device 20 is aligned with the respective device mounting openings 28 in either the first wall 10 or the second wall 14. In some aspects, the ion generator device 20 may be pre-wired before insertion. As described above, since the orientations of adjacent ion generator devices 20 may be reversed, every other ion generator device 20 may have the mounting tabs aligned with the device mounting openings 28 in the first wall 10 and every other ion generator device 20 may have the mounting tabs aligned with the device mounting openings 28 in the second wall 14. Once inserted, the resiliency of the rotated walls 10 and/or 14 may cause the walls 10, 14 to rotate back when the pressure is no longer asserted.

The ion generator devices 20 may be fixed in position using any suitable connecting hardware positioned and fastened into the mounting tabs and the device mounting openings 28. For example, the connecting hardware may include, but is not limited to, screws, bolts, rivets and nails.

In some aspects, the wires from the ion generator device 20 may be routed via the wire channel 400 to the strain relief fitting 24 in the endcap 18. The wiring may be routed before the endcap 18 is removably mounted.

In some aspects of the disclosure, to further prevent lateral movement of the ion generator device 20 within the fixture 1 and maintain the position of the first wall 10 with respect to the second wall 14, connecting hardware may also be positioned and fastened into hardware openings 30.

In an aspect of the disclosure, when maintenance of the fixture 1 is needed, such as when there is a visual indication through the opening 16 of a malfunction in one or more of the ion generator devices 20, the malfunctioned ion generator device 20 may be simply removed from the fixture 1. For example, the connected hardware associated with the malfunctioned ion generator device 20 may be removed from the device mounting openings 28 and if needed, the connecting hardware adjacent to the malfunctioned ion generator device 20 may also be removed from the hardware openings 30. Once removed, the malfunctioned ion generator device 20 may be removed from the fixture 1 by sliding the same out and disconnecting the wiring from the connector 405 and a new ion generator device may be mounted in the fixture 1.

In an aspect of the disclosure, the fixture 1 may have different lengths in the longitudinal direction. In some aspects, the length of the fixture 1 may be based on the length of the air conduit 605 which the fixture 1 will be mounted to. Additionally, the length of the fixture 1 may be based on the target airflow treated (such as cubic feet per minute). In some aspects of the disclosure, the fixture 1 may be about 1 foot. In other aspects of the disclosure, the fixture 1 may be about 12 feet. In other aspects of the disclosure, the fixture 1 may be between 1 foot and 12 feet. In some aspects, the fixture 1 may be less than about 1 foot. In other aspects, the fixture 1 may be more than about 12 feet.

In an aspect of the disclosure, the fixture 1 may hold one or more ion generator devices 20. The number of ion generator devices 20 in a fixture 1 may be based on the length of the fixture 1 and volume of the target airflow for treated. In an aspect of the disclosure, when multiple ion generator devices 20 are mounted within the fixture 1, the ion generator devices 20 may be equally spaced within the fixture 1 the longitudinal direction. This equal spacing may uniformly provide ions within the air conduit 605 when the fixture 1 is mounted to or in the air conduit 605. Additionally, the equal spacing may further reduce a likelihood of opposite charged ions from re-combining where the ion generator device 20 provides positive ions and negative ions.

In some aspects of the disclosure, the equal spacing is about 1 ft apart on center in the longitudinal direction. For example, there may be 8 ion generator devices 20 in an 8 foot fixture 1.

In an aspect of the disclosure, the fixture 1 may be formed from a plurality of fixture sections or fixture modules. In some aspects, the fixture sections or fixture modules may be welded together. In other aspects of the disclosure, the fixture sections or fixture modules may be removably attached to each other using a suitable attachment means. For example, the fixture sections or fixture modules may be connected to each other such as, but not limited to, using a mounting plate, mounting clips, adhesives, magnetic connectors, straps.

By having multiple sections or modules which are removably attached to each other, maintenance of the fixture may be easier. For example, in a case when maintenance of the fixture 1 is needed, such as when there is a visual indication through the opening 16 of a malfunction in one or more of the ion generator devices 20, the section or module of the fixture 1 having the malfunctional ion generator devices 20 may be removed and replaced with a new section or module simply by detaching a section or module.

In an aspect of the disclosure, the fixture 1 may be mounted in different orientations and different locations within the air conduit 605 or air flow system. The fixture 1 may comprise a adjustable mount(s) 22. In some aspects of the disclosure, the adjustable mount(s) 22 may be mounting brackets. The number and position of the adjustable mount(s) 22 (in the longitudinal direction) may be based on the length of the fixture 1 (and number of fixture sections or fixture modules). Additionally, the number of adjustable mount(s) 22 and position (in the longitudinal direction) may be based on the type of air conduit 605 in which the fixture 1 is mounted to or in.

As shown in the figures, the adjustable mount(s) 22 are located at the ends of the fixture 1 in the longitudinal direction; however, the adjustable mount(s) 22 may be located toward the middle of the fixture 1 as needed (or both). The adjustable mount(s) 22 may be aligned with the hardware openings 30 in the first wall 10 and the second wall 14 for attachment to the fixture 1.

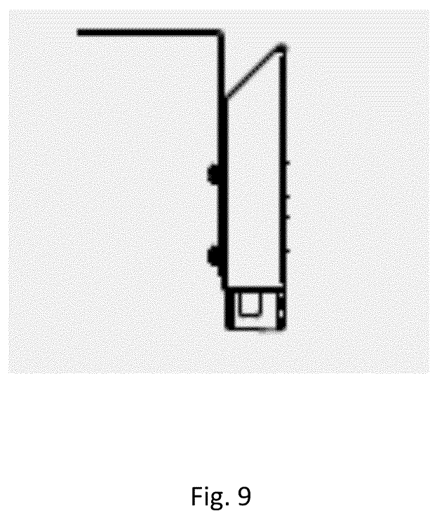

The figures also show the adjustable mount(s) 22 attached to the first wall 10 and the second wall 14 such that the fixture 1 may be mounted in or to the air conduit 605 in a horizontal orientation (an example of a first orientation). To change a target mounting orientation from the horizontal orientation to a vertical orientation (an example of a second orientation), the connecting hardware securing the adjustable mount(s) 22 to the walls 10, 14 may be loosened (to unfasten from the hardware openings 30). The adjustable mount(s) 22 may then be moved and the connecting hardware is re-fastened to the hardware openings 30 to secure the adjustable mount(s) 22 in the new position. In some aspects, the movement may be a rotation. FIG. 9 illustrates an example of the position of the adjustable mount(s) 22 to support a vertical orientation. In some aspects, the different orientations may be orthogonal. However, the different orientations are not limited thereto.

A vertical orientation may be a target installation orientation where there is a limited space for the fixture 1 in the direction of the airflow. This may be based on a specific airflow system. For example, a space in the air flow direction between an air filter and the heating or cooling coil(s) 600 may be narrow.

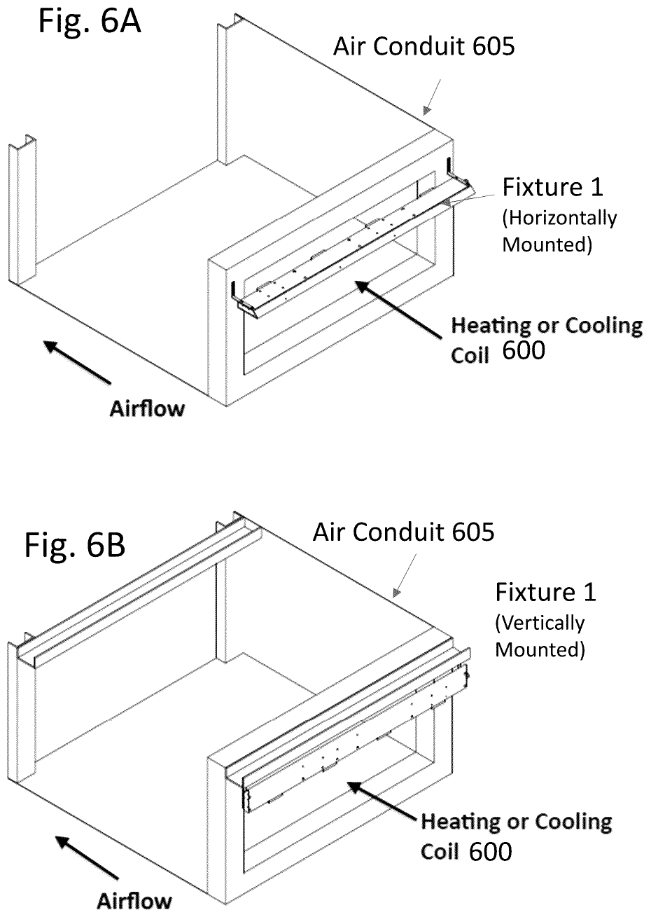

In some aspects of the disclosure, the fixture 1 may be mounted upstream of the heating or cooling coil(s) 600 in the air conduit 605 such as shown in FIG. 6A or 6B (and downstream of the air filters). This mounting location enables the ions to flow through the heating or cooling coil(s) 600 reducing the accumulation of contaminants (such as films and other pollutants) on the surface of the heating or cooling coil(s) 600. When mounting the fixture 1 in horizontal orientation, the fixture 1 may be directly mounted to the frame of the heating or cooling coil(s) 600. The fixture 1 may be mounted using suitable connecting hardware, such as, but not limited to, similar connecting hardware described above.

When mounting the fixture 1 in a vertical orientation, the fixture 1 may be mounted to a support structure 700 (which is attached to the frame for the heating or cooling coil(s) 600). Using a support structure 700 may enable a mounting which avoids mounting to the top of the air conduit 605. While mounting the fixture 1 directly to the top of the air conduit 605 may be possible, depending on the structure of the air conduit (such as a single or multiple layer element) it may create an opening in the air conduit 605 which may let in moisture and debris.

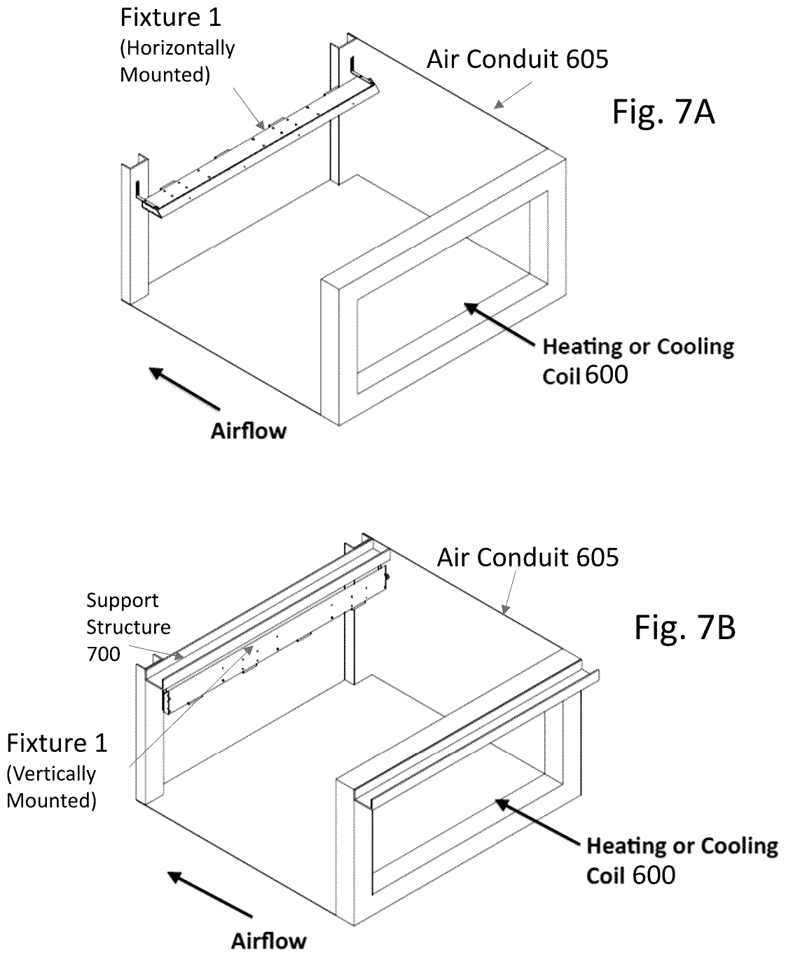

In some aspects of the disclosure, the fixture 1 may be mounted downstream of the heating or cooling coil(s) 600 in the air conduit 605 such as shown in FIG. 7A or 7B (and downstream of the air filters). In some aspects, this location may also be downstream of the fan/blower. This mounting location may provide ions closer to the occupied space than an upstream mount. Similar to above, when mounting the fixture 1 in a vertical orientation, the fixture 1 may be mounted to a support structure 700 (which is attached to the air conduit 605). Also, when mounting the fixture 1 downstream in a horizontal orientation, a support structure may be used to connect the fixture 1 to the air conduit 605. The support structure may be any shape or size and may depend on the shape/size of the air conduit 605.

In other aspects, the fixture 1 may be mounted upstream of both an air filter and the heating or cooling coil(s) 600. This position provides ions to the filter which deactivates contaminants captured by the filter. The life of the filter may be extended by this placement.

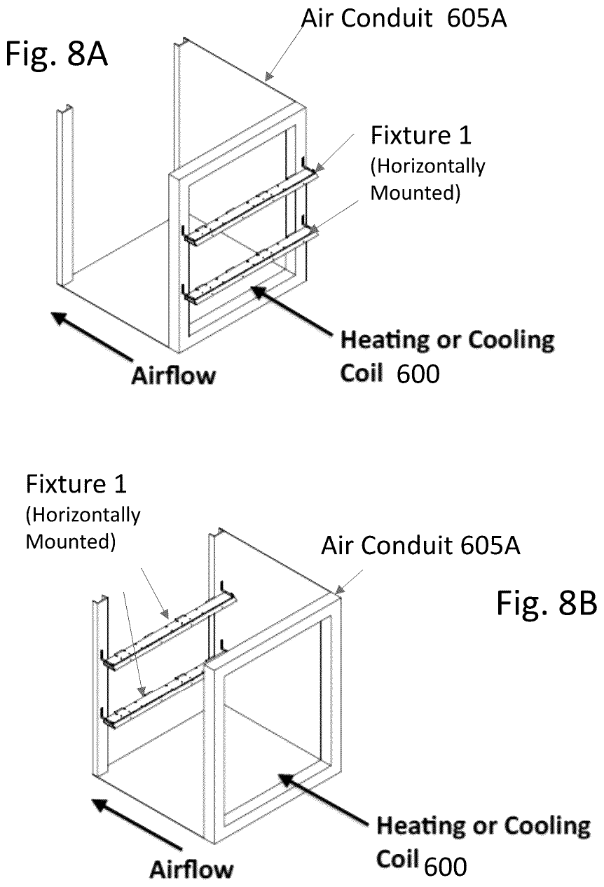

In some aspects of the disclosure, multiple fixtures 1 may be mounted in or to an air conduit 605 depending on the size of the air conduit 605 or treatment space. When multiple fixtures 1 are mounted, the fixtures 1 may be vertically aligned. FIGS. 8A and 8B illustrate an air conduit 605A having multiple fixtures 1 mounted in or thereto (in FIG. 8A, two fixtures 1 are mounted upstream of the heating or cooling coil(s) 600 and in FIG. 8B, two fixtures 1 are mounted downstream of the heating or cooling coil(s) 600). The number of fixtures 1 is not limited to 2 and FIGS. 8A and 8B are only provided as examples and any number of fixtures 1 may be included. Additionally, when multiple fixtures 1 are used, the mounting orientation is not limited to horizontal. Other mounting orientations may be used. For example, one of the fixtures may be mounted with the ionizing elements facing downward and another fixture 1 may be mounted with the ionizing elements facing upward. The fixtures 1 do not need to be mounted in the same mounting orientation.

In other aspects of the disclosure, the adjustable mount(s) 22 may be omitted and the fixture 1 may be mounted directly on or to the air conduit 605 such as via an adhesive, strap, magnetic connectors, clip, or any other suitable connector in either the horizontal orientation or vertical orientation. For example, the first wall 10 or the second wall 14 may be attached to the top or bottom of the air conduit 605 via an adhesive. Additionally, the fixture 1 may be fixed to the frame of the heating or cooling coil(s) 600 using a screw, nail, bolt, etc. inserted into the hardware opening 30 and directly into the frame. The fixture 1 may also be directly fixed to the frame using magnetic connectors or contacts.

In an aspect of the disclosure, the front face of each of the ion generator devices 20 may be aligned as viewed from the longitudinal direction. However, in other aspects of the disclosure, the front face may be staggered.

As shown in FIG. 2, the front of the fixture 1 does not have a wall, e.g., opened. However, in other aspects of the disclosure, the fixture 1A may comprise a fourth wall 1000 (front wall) (not including the endcaps 18) such as shown in FIG. 10. The fourth wall 1000 may be a sectional wall. The sections of the fourth wall may be between the ion generator device 20. In some aspects, this fourth wall 1000 may only extend partially between the first wall 10 and the second wall 14. For example, the fourth wall 1000 may extend from the first wall 10 or the second wall 14 and there may be a gap between the other wall (in the top-down direction). In other aspects, the fourth wall 1000 may extend the entire length from the first wall 10 to the second wall 14 (in the top-down direction) such as shown in FIG. 10. The sections of the fourth wall may further limit the lateral movement of each ion generator device 20. Additionally, the sections of the fourth wall may also prevent ions from the ion generator device 20 from travelling into the fixture 1A. The sections of the fourth wall may also provide further structural integrity to the fixture 1A.

In some aspects of the disclosure, each ion generator device 20 may be self or auto cleaning. This means that the ion generator device 20 periodically cleans debris (such as dust and dirt) from the ion generating elements.

In the discussion and claims herein, the term “about” indicates that the value listed may be somewhat altered, as long as the alteration does not result in nonconformance of the process or device. For example, for some elements the term “about” can refer to a variation of ±0.1%, for other elements, the term “about” can refer to a variation of ±1% or ±10%, or any point therein. For example, the term about when used for a measurement in mm, may include +/0.1, 0.2, 0.3, etc., where the difference between the stated number may be larger when the stated number is larger. For example, about 1.5 may include 1.2-1.8, where about 20, may include 19.0-21.0.

As used herein, the term “substantially”, or “substantial”, is equally applicable when used in a negative connotation to refer to the complete or near complete such a lack of an action, characteristic, property, state, structure, item, or result. For example, a surface that is “substantially” flat would either be completely flat, or so nearly flat that the effect would be the same as if it were completely flat. “Substantially” when referring to a shape or size may account for manufacturing tolerances where a perfect shapes, edges, turns, such as circular or sizes or angles may be difficult to manufacture.

References in the specification to “one aspect”, “certain aspects”, “some aspects” or “an aspect”, indicate that the aspect(s) described may include a particular feature or characteristic, but every aspect may not necessarily include the particular feature, structure, or characteristic. Moreover, such phrases are not necessarily referring to the same aspect. Further, when a particular feature, structure, or characteristic is described in connection with an aspect, it is submitted that it is within the knowledge of one skilled in the art to affect such feature, structure, or characteristic in connection with other aspects whether or not explicitly described.

The described aspects and examples of the present disclosure are intended to be illustrative rather than restrictive, and are not intended to represent every aspect or example of the present disclosure. While the fundamental novel features of the disclosure as applied to various specific aspects thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or aspects of the disclosure may be incorporated in any other disclosed or described or suggested form or aspects as a general matter of design choice. Further, various modifications and variations can be made without departing from the spirit or scope of the disclosure as set forth in the following claims both literally and in equivalents recognized in law.

What is claimed is:

1. A fixture configured to retain one or more ion generator devices, each ion generator device being configured to generate positive ions, negative ions or both positive and negative ions, each ion generator device comprises a status indicator, the fixture comprising:
a first wall;
a second wall opposing the first wall; and
a connecting wall connecting the first wall and the second wall, the first wall, the second wall or the connecting wall having one or more openings,
wherein when the one or more ion generator devices is retained in the fixture, the first wall and the second wall sandwich the one or more ion generator devices, and light emitted or not emitted from a status indicator of a respective ion generator device is viewable from outside the fixture via a corresponding opening.

2. The fixture of claim 1, wherein the connecting wall is inclined with respect to the second wall.

3. The fixture of claim 1, wherein the inclination is about 45°.

4. The fixture of claim 1, wherein the connecting wall has the one or more openings.

5. The fixture of claim 1, further comprising a protective film or lens inserted into each opening.

6. The fixture of claim 1, wherein the first wall, the second wall and the connecting wall provide a channel configured for electrical wiring to be routed from an external control panel to each of the one or more ion generator devices.

7. The fixture of claim 1, wherein the first wall, the second wall and the connecting wall are formed from a single sheet of material.

8. The fixture of claim 1, wherein the one or more ion generator devices is a plurality of ion generator devices, wherein when the plurality of ion generator devices is retained in the fixture, the plurality of ion generator devices is aligned.

9. The fixture of claim 1, wherein the fixture is configured to be mounted in a first orientation or a second orientation, where the first orientation is different from the second orientation.

10. The fixture of claim 9, further comprising a plurality of mounting brackets, wherein when the fixture is to be mounted in the first orientation, the plurality of mounting brackets is in a first position and wherein when the fixture is to be mounted in the second orientation, the plurality of mounting brackets is moved to a second position.

11. The fixture of claim 9, wherein the fixture is configured to be mounted in one of the first orientation or the second orientation downstream of a heating or cooling coil of a heating, ventilating, and air-conditioning system (HVAC).

12. The fixture of claim 1, wherein the fixture is configured to be mounted in an air handling unit (AHU), an air duct, a rooftop unit (RTU), a dedicated outdoor air system (DOAS), packaged Terminal Air Conditioners (PTAC) or a makeup air unit (MAU).

13. The fixture of claim 1, wherein the one or more ion generator devices is a plurality of ion generators and each ion generator device comprises two electrodes and is configured to generate both positive and negative ions, where the positive ions are generated from a first electrode and the negative ions are generated from a second electrode, wherein when mounted in the fixture, adjacent ion generator devices are in different orientations such that a first electrode is adjacent to another first electrode or a second electrode is adjacent to another second electrode.

14. The fixture of claim 1, further comprising a first endcap and a second endcap positioned at respective ends in a longitudinal direction.

15. The fixture of claim 14, wherein the first endcap has an opening configured to receive electrical wiring to be routed to each of the one or more ion generator devices, wherein the electrical wiring is connected to an external control panel, where the external control panel comprises a power source.

16. The fixture of claim 15, further comprising a strain relief fitting inserted into the opening of the first endcap, wherein the strain relief fitting surrounds the electrical wiring.

17. The fixture of claim 1, a length in a longitudinal direction is between about 1 foot to about 12 feet.

18. The fixture of claim 1, further comprising a sectional fourth wall extending between the first wall and the second wall and between the one or more ion generator devices.

19. The fixture of claim 18, wherein the sectional fourth wall extends from the first wall to the second wall.

20. A fixture configured to retain one or more ion generator devices, each ion generator device being configured to generate positive ions, negative ions or both positive and negative ions, the fixture comprising:
a first wall;
a second wall opposing the first wall; and
a connecting wall connecting the first wall and the second wall,
wherein the first wall, the second wall or the connecting wall have one or more openings, and the first wall, the second wall or the connecting wall have one or more status indicators, and
wherein when the one or more ion generator devices is retained in the fixture, the first wall and the second wall sandwich the one or more ion generator devices, and each of the one or more ion generator devices is connected to a corresponding one of the one or more status indicators by a respective connection cable routed through an opening or the one or more status indicators is mounted in the one or more openings.

21. A fixture configured to retain one or more ion generator devices, each ion generator device being configured to generate positive ions, negative ions or both positive and negative ions, the fixture comprising:
a first wall;
a second wall opposing the first wall;
a connecting wall connecting the first wall and the second wall; and
a sectional fourth wall extending between the first wall and the second wall and between the one or more ion generator devices, the sectional fourth wall having one or more opening and one or more status indicators,
wherein when the one or more ion generator devices is retained in the fixture, the first wall and the second wall sandwich the one or more ion generator devices, and each of the one or more ion generator devices is connected to a corresponding one of the one or more status indicators by a respective connection cable routed through an opening or the one or more status indicators is mounted in the one or more openings.
